(19) United States
(12) Patent Application Publication
(10) Pub. No.: US 2024/0268934 A1
(43) Pub. Date: Aug. 15, 2024

(54) Offset IntraOral Cavity Scanner

(71) Applicant: Jeffery A. Scolnick, New York, NY (US)

(72) Inventor: Jeffery A. Scolnick, New York, NY (US)

(21) Appl. No.: 18/225,146

(22) Filed: Jul. 23, 2023

Related U.S. Application Data
(60) Provisional application No. 63/444,596, filed on Feb. 10, 2023.

(51) Int. Cl. A61C 9/00; A61B 1/247

(52) U.S. Cl. CPC A61C 9/0053; A61B 1/247

(57) ABSTRACT

An oral cavity scanner is presented that includes a scanning head offset from an axis of the scanner that allows for a single pass accurate representation of teeth within the oral cavity. In one aspect of the invention, the scanning head, which is removably attachable to the scanner, includes a light director that presents a true image of an object being viewed to the scanning elements of the scanner.

### CLAIM OF PRIORITY

This application claims, pursuant to 35 USC 119, priority to, and the benefit of, the earlier filing date of provisional patent application afforded Ser. No. 63/444,596, filed on Feb. 10, 2023, the contents of which are incorporated by reference, herein.

### BACKGROUND OF THE INVENTION

### Field of the Invention

This invention relates to the field of dentistry and more particularly to a scanner for the improved prosthetic dental mapping of teeth and surrounding tissue.

### Background Information

Dental care has become increasingly important as medical science has traced many types of infections to health of the gums and teeth within the oral cavity. Teeth replacement is of particularly importance since the failure to replace lost teeth alters the health of a person, in failing to eat certain foods, while causing changes within the jaw as the remaining teeth may shift to the areas of the lost teeth.

A first method for capturing a representation of a patient's teeth in preparing to insert new teeth into the patient's oral cavity is to form a model of a patient's teeth using a moldable material that conforms to the patient's teeth. From the impressions within the moldable material, construction of a replacement tooth or a bridge, spanning to two teeth, may be obtained. However, this method is both cumbersome and quite distasteful to the patient. Another method used to form a representation of the patient's teeth is to scan the patient's oral cavity using an oral scanner. In this method a light is shone onto the patient's teeth and images of the patient's teeth (from the reflection of the light) are then captured by a camera or other similar type image capture devices. A representation of the patient's teeth may then be formulated using special purpose software from the capture images from which replacement teeth and/or bridges may be developed.

Generally, these oral scanners, while accurate in producing a three-dimensional (3D) model of the patient's teeth, require multiple passes of the patient's teeth as these scanners are designed for the viewing of the surfaces of the patient's teeth directly below the scanners field of view. See for example, such as that disclosed in US 2020/0170497.

To obtain an accurate 3D representation of the teeth structure, multiple passes (i.e., top, exterior side, interior side) of the teeth are required, wherein the images are joined together externally.

However, requiring multiple passes of the same region within the mouth to obtain accurate images for modeling is time consuming and further may introduce a gag reflex on the part of the patient.

Hence, there is a need in the industry for a device suitable for the scanning and capturing of images of a patient's teeth in a fast and easy manner in order to present a more accurate representation that may be used in the formation of replacement teeth and/or bridge work.

### SUMMARY OF THE INVENTION

Disclosed is an oral scanner that is adapted to collect data or images associated with a patient's teeth in order to develop a more accurate model of the patient's oral cavity.

Disclosed is an oral scanner that includes a head element that is suitable for insertion along a side of a patient's teeth to capture data or images to develop a more accurate model of the patient's oral cavity.

Disclosed is a dental oral scanner that may include interchange heads, one which is suitable for insertion into an oral cavity to capture date or image to develop a more accurate of the patient's teeth and/or oral cavity structure.

Disclosed is a dental oral scanner wherein the scanning head is oriented substantially ninety degrees with respect to the orientation of the scanner.

Disclosed is an exemplary embodiment of a dental scanner in accordance with the principles of the invention.

For a better understanding of exemplary embodiments and to show how the same may be carried into effect, reference is made to the accompanying drawings. It is stressed that the particulars shown are by way of example only and for purposes of illustrative discussion of the preferred embodiments of the present disclosure and are presented to clarify the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

### BRIEF DESCRIPTION OF THE DRAWINGS

The advantages, nature, and various additional features of the invention will appear more fully upon consideration of the illustrative embodiments described in detail in connection with the accompanying drawings, where like or similar reference numerals are used to identify like or similar elements throughout the drawings:

FIG. 1 illustrates a perspective view of a conventional intraoral cavity scanner for creating a representation of the teeth of a patient.

FIG. 2 illustrates a perspective view of a first exemplary embodiment of an intraoral cavity scanning device in accordance with the principles of the invention.

FIG. 3 illustrates a perspective view of a second exemplary embodiment of an offset intraoral scanning device in accordance with the principle of the invention.

FIG. 4 illustrates a perspective view of another aspect for implementing a light transformer in accordance with the principles of the invention.

FIG. 5 illustrates a perspective view of a still another aspect for implementing light transformer in accordance with the principles of the invention.

FIG. 6 illustrates a top view of an exemplary embodiment of an offset intraoral cavity scanner head shown in FIGS. 3 and 4 in accordance with the principles of the invention.

FIG. 7 illustrates a perspective view of the scanner head shown in FIG. 6.

FIG. 8 illustrates an exploded perspective view of an exemplary embodiment of an offset intraoral scanning device in accordance with the principles of the invention.

It is to be understood that the figures, which are not drawn to scale, and descriptions of the present invention described herein have been simplified to illustrate the elements that are relevant for a clear understanding of the present invention, while eliminating, for purposes of clarity, many other elements. However, because these omitted elements are well-known in the art, and because they do not facilitate a better understanding of the present invention, a discussion of such elements are not provided herein. The disclosure, herein, is directed also to variations and modifications known to those skilled in the art.

### DETAILED DESCRIPTION OF THE INVENTION

As used herein, the terms “comprises”, “comprising”, “includes”, “including”, “has”, “having”, or any other variation thereof, are intended to cover non-exclusive inclusions. For example, a process, method, article or apparatus that comprises a list of elements is not necessarily limited to only those elements but may include other elements not expressly listed or inherent to such process, method, article, or apparatus. In addition, unless expressly stated to the contrary, the term “of” refers to an inclusive “or” and not to an exclusive “or”. For example, a condition A or B is satisfied by any one of the following: A is true (or present) and B is false (or not present); A is false (or not present) and B is true (or present); and both A and B are true (or present).

The terms “a” or “an” as used herein are to describe elements and components of the invention. This is done for convenience to the reader and to provide a general sense of the invention. The use of these terms in the description, herein, should be read and understood to include one or at least one. In addition, the singular also includes the plural unless indicated to the contrary. For example, reference to a composition containing “a compound” includes one or more compounds. As used in this specification and the appended claims, the term “or” is generally employed in its sense including “and/or” unless the content clearly dictates otherwise.

All numeric values are herein assumed to be modified by the term “about,” whether or not explicitly indicated. The term “about” generally refers to a range of numbers that one skill in the art would consider equivalent to the recited value (i.e., having the same function or result). In any instance, the terms “about” may include numbers that are rounded (or lowered) to the nearest significant figure.

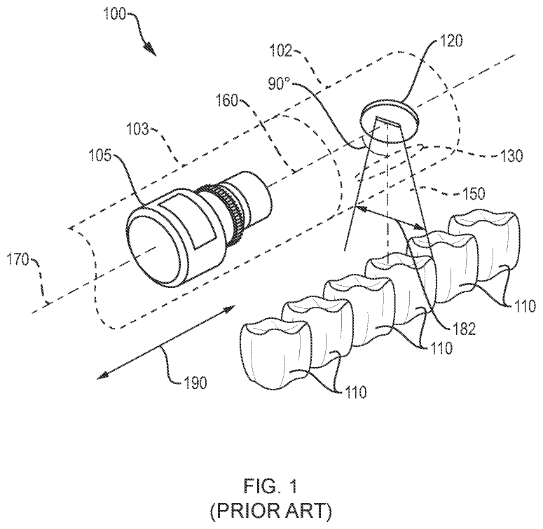

FIG. 1 illustrates a perspective view of a conventional intraoral cavity scanner for creating a representation of the teeth of a patient.

In this illustrated view, intraoral cavity scanner device 100 comprises a scanner head 102 that may be removably attachable to scanner body 103, wherein within scanner body 103 resides a scanner element 105, which is suitable of collecting images of an object, such as one or more teeth 110. In this illustrated example, object 110 is represented by a plurality of teeth arranged in a row contained within a patient's oral cavity.

Scanner element 105 may comprise scanning elements, such as a camera, a CMOS device or a CCD device (not shown), that are suitable for capturing images of objects 110. Although not shown, it would be recognized that scanner body 103 further may include a light source that provides an illumination to the illustrated objects 110.

Within scanner head 102 (referred to herein, interchangeably as scanner tip) is reflective element (e.g., mirror, highly polished metallic, highly polished plastic materials) 120. Reflective element 120, which is oriented at a substantially forty-five (45) angle to an optical axis 160 of intraoral cavity scanner device 100, provides for the capture of light, associated with objects 110, entering window 130. In the operation of intraoral cavity scanner device 100, images of objects (i.e., teeth) 110, viewed through window 130, along axis 150 are directed along optical axis 160 to scanner element 105. Accordingly, reflective element 120 provides for the transition of the orientation of light entering scanner device 100 from one of substantially perpendicular to scanner device 100 (i.e., along axis 150) to one of substantially parallel to scanner device 100, wherein optical axis 160 is substantially co-incident to longitudinal axis 170 of scanner head 102.

Images of objects 110 provided to scanner element 105 by the redirection of light by reflective surface 120, along optical axis 160 may then be recorded by the scanner element 105 within scanner body 103 as intraoral cavity scanner device 100 moves in the direction of travel 190.

The captured images of object 110 may then be provided to a computing system (not shown) that may utilize the captured images to create a 3D model of the patient's teeth. To assist in the preparation of a model, the intraoral cavity scanner device 100 may be pitched left and right along longitudinal axis 170 to obtain offset images of the objects 110.

In a more practical manner, however, to form an accurate model of objects (e.g., teeth) 110 within the oral cavity, multiple passes of objects 110 are necessary as intraoral cavity scanner device 100 is limited to providing scans of objects substantially perpendicular to scanner device 100. That is, intraoral cavity scanner device 100 is first moved in the direction of travel 190 to capture images of the top surfaces of objects 110 and then oriented substantially ninety (90) degrees, both left and right, to capture images of sides of objects 110 while moving in the direction of travel 190.

Specialized software within the not shown computing system is then used to “stitch” (i.e., merge) together the different images collected by the different scans of the same object(s) 110.

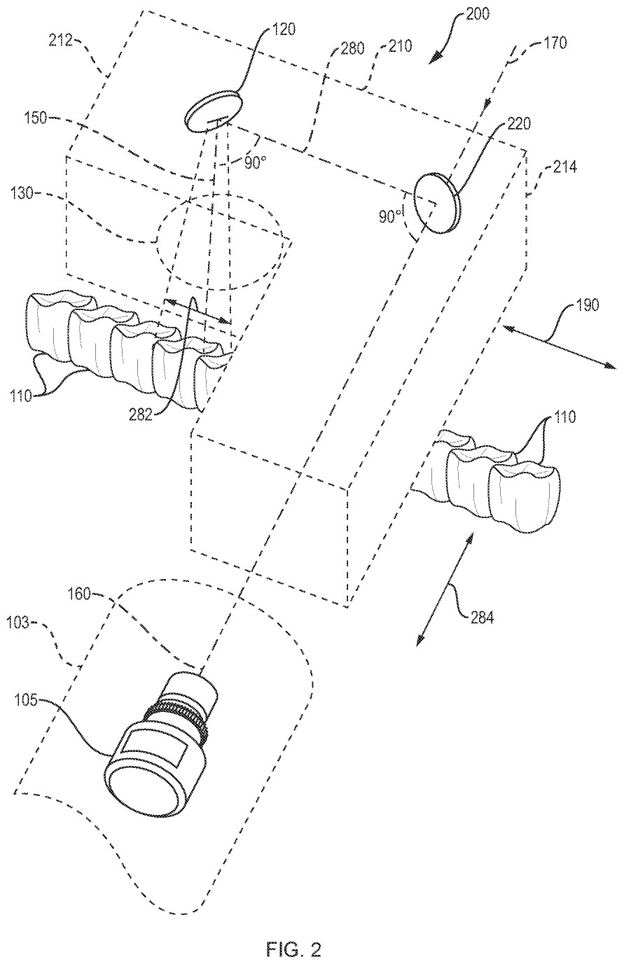

FIG. 2 illustrates a perspective view of a first exemplary embodiment of an intraoral cavity scanning device in accordance with the principles of the invention.

In this illustrated embodiment, intraoral cavity scanner device 200 comprises a scanner body 103 including scanner element 105 and scanning elements (not shown), similar to that shown in FIG. 1, and scanner head 210. Accordingly, a further discussion of the elements common to FIG. 1 need not be provided with regard to the device shown in FIG. 2, as it is believed one skilled in the art would obtain the knowledge or the understanding of these elements from the discussion made with regard to FIG. 1.

In accordance with the principles of the invention, scanner head 210 comprises a linear section 214 and an offset section 212, wherein linear section 214 is arranged along longitudinal axis 170, in a manner similar to that shown in FIG. 1.

Offset section 212, as shown, is oriented offset from linear section 214 and includes, therein, viewing window 130, through which objects (e.g., teeth) may be viewable, as discussed with regard to FIG. 1.

Although scanner head 210 is shown possessing an offset section 212 that is substantially perpendicular to linear section 214, it would be recognized that offset section 212 may be oriented from linear section 214 at an alternative angle without altering the scope of the invention. Thus, while the discussion, herein, refers to orientations of forty-five (45) degrees and redirection by ninety (90) degrees, it would be recognized that the orientation (offset) of offset section 212 with respect to linear section 214 may be different than that discussed and those skilled in the art would have the knowledge and understanding to alter the orientation and the re-orientation of the reflecting elements to redirect light to achieve the objects disclosed, herein, without undue experimentation. Thus, these alternative configurations have been contemplated and considered within the scope of the invention claimed.

In this first exemplary embodiment of an offset intraoral scanner device 200, offset intraoral cavity scanner device 200 includes a plurality reflective elements 120, 220 that are arranged to capture images of a patient's teeth 110, viewed through viewing port 130. Reflective element (e.g., mirror) 120, similar to that described with regard to FIG. 1, directs light associated with images of objects 110, which is obtained substantially perpendicular to scanner head 210, along axis 150, toward reflective element 220 along axis 280. In accordance with the illustrated aspect of the invention, reflective element 120 is oriented at a forty-five (45) degree angle with respect to axis 150 and concurrently oriented at a forty-five (45) degree angle with respect to axis 280. In this manner, light from objects 110, captured along axis 150 may be transitioned (i.e., redirected), at a substantially ninety (90) degree angle, along axis 280.

Reflective element 220 (i.e., mirror, highly reflective surfaces, etc.), receiving light associated with the images of objects 110 along axis 280 subsequently, based on the orientation of reflective element (e.g., surface) 220, redirects the received light, at a substantially ninety (90) degree along, toward scanner element 105, along optical axis 160.

Accordingly, as offset intraoral cavity scanner device 200 is moved, laterally with respect to objects 110, along axis 190 images of a top surface of objects 110. In addition, intraoral cavity scanner device 200 may concurrently be moved transversely with respect to object 110, along axis 284, to capture images of the sides of objects 110. The captured images (top and sides) may be used to create an accurate model of the objects 110 in a single pass.

The single pass of offset intraoral cavity scanner device 200 is advantageous over a conventional intraoral cavity scanner device 100 as it provides for a less intrusive means for the collection of the data needed to prepare a 3D model of a patient's teeth (objects 110)

Although, in the illustrated embodiment, reflective surfaces 120, 220 are shown at substantially a forty-five (45) degree angle with respect to offset section 212 and linear section 214, it would be recognized that the illustrated orientation (i.e., 450) of reflective surfaces 120, 220 is selected to allow light associated with images of objects 110 to be properly presented to scanner element 105. Thus, the orientation of reflective surfaces 120, 220 may be altered from the orientation shown, based, in part, on the orientation of offset section 212 with respect to linear section 214, such that light associated with images of objects 110 may be properly presented along axis 160 to scanner element 105.

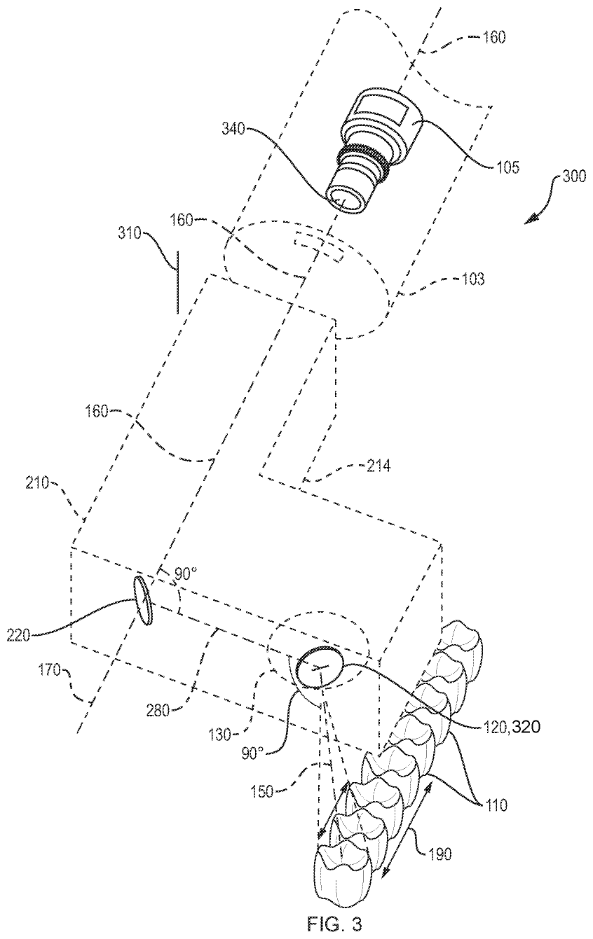

FIG. 3 illustrates a perspective view of a second exemplary embodiment of an offset intraoral scanning device in accordance with the principle of the invention.

In this illustrated aspect of the invention, offset intraoral cavity scanner device 300 comprises a scanner tip 310 comprising a linear section 214 and an offset section 212, wherein scanner tip 310 is removably attachable to scanner body 103.

In accordance with this embodiment of the invention, scanning tip 310 includes viewing window 130, which is oriented to capture light from objects 110 (along axis 150), as previously discussed with regard to FIGS. 1 and 2, wherein the received light is transitioned (or re-directed), by, in this illustrated case by ninety (90) degrees, along axis 280. In addition, included, within linear section 214, is reflective surface 220, which receives light, along axis 280, associated with images of objects 110 and redirects the light (by substantially ninety (90) degrees), along axis 160, toward scanner element 105 with scanner body 103.

In this illustrated aspect of the invention, scanner element 105 comprises one or more scanning elements 340 that convert the light associated with objects 110 into electrical signals that may be subsequently processed by computing systems (not shown) to form a 3D image of objects 110. Scanning elements 340 may comprise one or more of a charged coupled device, (CCD) a CMOS device, and a photo-detector device, which are well known in the art and a more detailed discussion of such well-known devices is believed not necessary for the understanding of the invention claimed.

The orientation of offset section 212 allows for the collection of images of objects 110 transverse to the direction of the direction of travels 190, 284, such that only a single pass of scanner device 300 is required to collect sufficient information to create an accurate model of the patient's teeth 110.

To further disclose different aspects of the offset intraoral cavity device claimed, reflective surface (element) 120 is further referred to as light transformer 320, wherein light transformer 320 “transforms” the orientation of light entering window 130 from a first direction (i.e., substantially perpendicular) to objects 110 to a second direction (i.e., substantially transverse to objects 110).

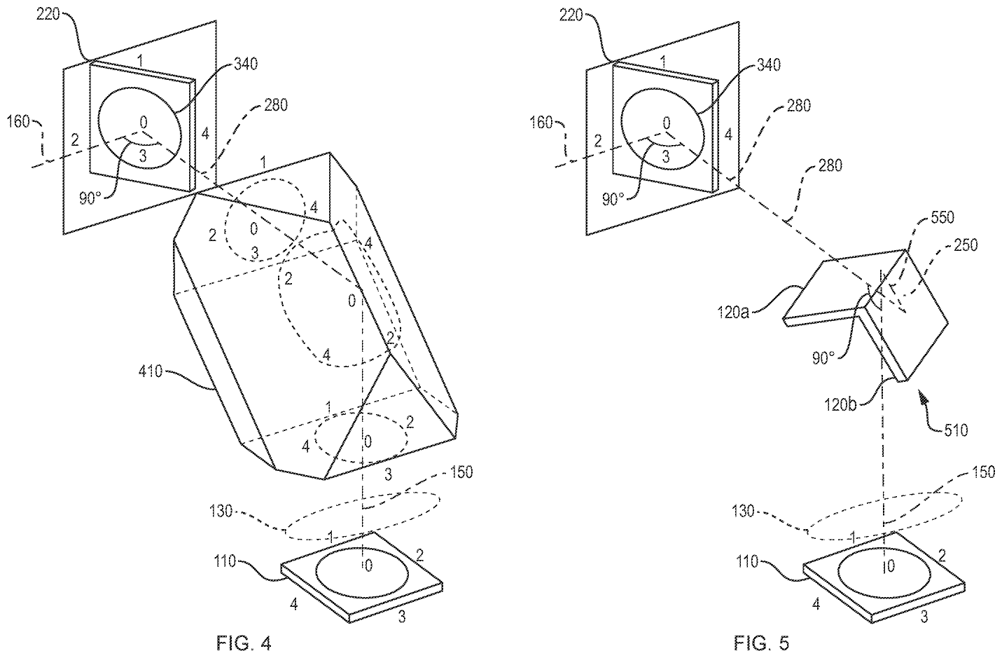

FIG. 4 illustrates a perspective view of another aspect for implementing a light transformer in accordance with the principles of the invention.

In this illustrated aspect of the invention, an Amici Roof Prism 410 is utilized as light transformer 320, wherein images associated with object 110, viewed through window 130, are inverted as light is redirected toward reflective surface 220.

Amici roof prisms are known in the art to be a reflecting prism used to deviate a beam of light by a desired number of degrees (in this illustrated example, 90 degrees) while simultaneously inverting the image through the total internal reflection of the light entering the prism. As shown in FIG. 4, the labels 1, 2, 3, 4, associated with edges of object 110, are shown in a first order on object 110 and shown in a reversed order on reflective surface 220 after passing through prism 410.

Although not shown, it would be recognized that reflective surface 220 further reverses the orientation of object 110 to present the image of object 110 in a correct orientation on scanning elements 340. Accordingly, the incorporation of Amici Roof Prism 410 as light transformer 320 provides for the viewing of a true image of the object viewed.

FIG. 5 illustrates a perspective view of a still another aspect for implementing light transformer in accordance with the principles of the invention.

In this illustrated aspect, a dual reflective surface element 510 is utilized as light transformer 320, wherein images associated with object 110 (i.e., labels 1, 2, 3, 4), viewed through window 130, are inverted such that the labels 1, 2, 3, 4 of the image presented to reflective surface 220 is reversed.

In this exemplary aspect, dual reflective surface element 510 comprises two substantially highly polished surfaces, which in this illustrated example are referred to as 120a, 120b,similar to element 120, arranged substantially perpendicular (i.e., ninety (90) degrees) to each other. In this manner, light associated with images of object 110, directed along axis 150, viewed through window 130, is directed toward surface 120a, which redirects the received light along axis 550 toward reflective surface 120b. Reflective surface 120b, in receiving light along axis 550, redirects the received light along optical axis 280 toward reflective surface 220.

In accordance with this aspect of the invention, images associated with objects 110 are reversed by the light transformer 320 and reversed again by reflective surface 220 so as to present a correct orientation of objects 110 to scanning elements 340.

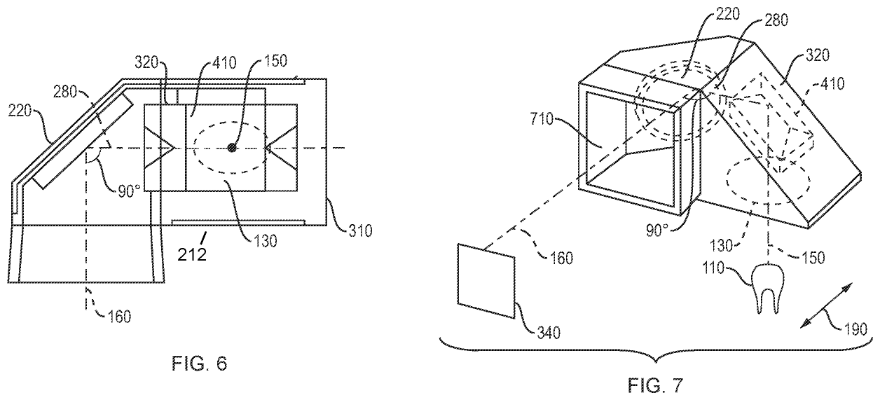

FIG. 6 illustrates a top view of an exemplary embodiment of an offset intraoral cavity scanner head 310 shown in FIGS. 3 and 4 in accordance with the principles of the invention.

In this illustrated view, scanner head 310 comprises window 130 (pointing downward into the plane of the paper) within offset section 212, through which an object (not shown) may be viewed along axis 150 (represented as a point positioned substantially centered within window 130). Further illustrated is Amici prism 410, representing light transformer 320, which is configured to re-direct light entering window 130, along axis 280, toward reflective element 220. Reflective element or surface 220 redirects the light received, along axis 280, along optical axis 160 toward scanner element 340 (not shown), as previously discussed.

In accordance with the principles of the invention, light associated with images of object 110 are first correctly oriented top-to-bottom by light transformer 320 (see FIGS. 4 and 5) and then correctly oriented left-to-right by reflective surface 220. Hence, the light associated with images of object 110 are presented to scanning elements 340 (e.g., camera, charge coupled device (CCD) or CMOS) along optical axis 160 as a true image of objects 110.

FIG. 7 illustrates a perspective view of the scanner head 310 shown in FIG. 6.

In this illustrated view is shown window 130 positioned above object 110, wherein light associated with object 110 is captured and redirected by light transformer 320 (e.g., Amici prism 410), toward reflective surface 220, wherein the light is further re-directed, along axis 160, toward opening 710. The light exiting opening 710 along axis 160 is directed to scanning elements 340 (not shown).

As previously discussed, the light associated with objects 110 is first inverted top-bottom by light transformer 320 and then inverted left-to-right by reflective surface 220 to present a true image to scanning elements 340.

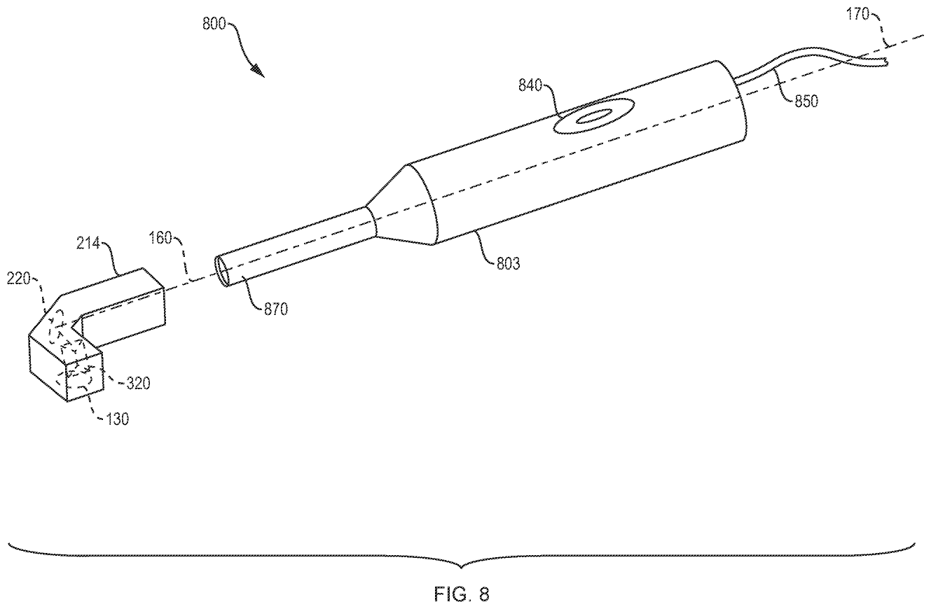

FIG. 8 illustrates an exploded perspective view of an exemplary embodiment of an offset intraoral scanning device in accordance with the principles of the invention.

In this illustrated embodiment is shown, offset intraoral scanning device 800 comprises scanning handle 803 (similar to handle 103, FIG. 1) including an on/off switch 840 that may be used to provide power to one or more electrical/electronic components (e.g., scanner element 105 and scanning elements 340, not shown) within handle 803. Further illustrated is scanning head 310 that may be removably attachable to handle 803, as previously discussed. Scanning head 310 comprises linear section 214 and offset section 212, wherein linear section 214 is substantially co-incident (i.e., parallel) with longitudinal axis 170 and offset section 212 oriented, in this illustrated example, substantially perpendicular to longitudinal axis 170.

Further illustrated is viewing window 130, positioned within offset section 212, wherein light associated with objects 110 (not shown) is directed toward window 130 along axis 150. Furthermore, tip 310 is further illustrated is light transformer 320 and reflective surface 220 that redirects light associated with object 110 (not shown) along axis 160 to present a correctly oriented image of object 110 to scanning elements 340 (not shown).

In one aspect of the invention, handle 805 may include an extension wand 810 onto which scanning head 310 may be removably attachable. Extension wand 810 may comprise an optical clear material that transfers light, exiting scanning head 310, toward scanning elements 340 (not shown). Alternatively, extension wand 810 may comprise a plurality of optical fibers that may transfer light, exiting scanning head 310, toward scanning elements 340.

In one aspect of the invention, extension wand 870 may be composed of a rigid material (e.g., hollow plastic tubing) that may be incorporated into offset intraoral scanner device 800, which allows for the removable attachment of scanning head 310, with respect to handle 805. Alternatively, extension wand 870 may be removably attachable to handle 805 to allow for different configurations of offset intraoral scanner device 800.

In one aspect of the invention, offset intraoral scanner device 800 may be provided electrical energy, which is used to power electronic components associated with scanner element 105, for example, using an electrical cord 850. Alternatively, intraoral scanner device 800 may comprise a battery compartment (not shown) for the storage of one or more batteries, wherein the batteries may provide electrical energy to electronic components associated with scanner element 105.

In summary, an offset intraoral cavity scanner device and scanner head assembly is presented that includes a scanning head oriented substantially perpendicular to an axis of the scanner device, wherein the scanning head includes a light director section that is used to present a true image of an object being viewed to scanning elements within the scanner device.

The invention has been described with reference to specific embodiments. One of ordinary skills in the art, however, appreciates that various modifications and changes can be made without departing from the scope of the invention as set forth in the claims. Accordingly, the specification is to be regarded in an illustrative manner, rather than with a restrictive view, and all such modifications are intended to be included within the scope of the invention.

Benefits, other advantages, and solutions to problems have been described above regarding specific embodiments. The benefits, advantages, and solutions to problems, and any element(s) that may cause any benefits, advantages, or solutions to occur or become more pronounced, are not to be construed as a critical, required, or an essential feature or element of any or all of the claims.

What is claimed is:

1. An intraoral cavity scanning device comprising:
a body section comprising:
a scanning element; and
a scanner head section, wherein said scanner head section comprises:
a linear section, substantially in-line with said body section; and
a offset section offset from said linear section, said offset section comprises a viewing window, wherein said offset section comprises:
a light transformer configured to:
capture light associated with images of an object viewed through said viewing window along a first axis;
re-orient, in a first direction, said captured light; and
provide said re-oriented light along a second axis, and
said linear section comprises:
a reflective surface configured to:
receive said re-oriented light along said second axis;
re-orient, in a second direction, said re-oriented light; and
provide said re-oriented light to said scanning element, wherein said provided re-oriented light provides a true image to said scanning element.

2. The scanning device according to claim 1, wherein said scanner head section is removably attachable to said body section.

3. The scanning device according to claim 1, wherein said light transformer comprises:
an Amici roof prism.

4. The scanning device according to claim 1, wherein said light transformer comprises:
dual reflective surfaces oriented substantially perpendicular to each other.

5. The scanning device according to claim 1, wherein said reflective surface is one of: a mirror, a highly polished metallic surface, and a highly polished plastic surface.

6. A scanner head for an intraoral scanning device comprising:
a offset section, said offset section comprises:
a viewing window; and
a light transformer, said light transformer configured to:
capture light associated with images of an object viewed through said viewing window along a first axis;
reverse, in a first direction, an orientation of said captured light; and
provide said reversed oriented light along a second axis, and
a linear section, offset from said offset section, said linear section comprising:
a reflective surface configured to:
receive said reverse oriented light along said second axis;
reverse, in a second direction, an orientation of said reverse oriented light; and
provide said light reversed in said first direction and in said second direction, along a third axis, wherein said image of said object along said third axis is presented as a true image of said object.

7. The scanner head according to claim 6, wherein said light transformer comprises:
an Amici roof prism.

8. The scanner head according to claim 6, wherein said light transformer comprises:
dual reflective surfaces oriented substantially perpendicular to each other.

9. The scanner head according to claim 6, wherein said reflective surface is one of: a mirror, a highly polished metallic surface, and a highly polished plastic surface.

10. The scanner head according to claim 6, wherein said scanner head is removably attachable to a scanner body of said intraoral scanning device.
